(19) United States
(12) Patent Application Publication
(10) Pub. No.: US 2008/0004192 A1
(43) Pub. Date: Jan. 3, 2008

(54) Composition

(75) Inventors: Venkateswara Kumar Vedantam, Paramus, NJ (US); Francois Bouton, Shanghai (CN)

Correspondence Address: Andrew N. Parfomak;Norris McLaughlin & Marcus, PA, 875 Third Avenue, 18th Floor, New York, NY, 10022, US

(73) Assignee: Givaudan SA, Vermier (CH)

(21) Appl. No.: 11/572,933

(22) PCT Filed: Jul. 29, 2005
(86) PCT No.: PCT/CH05/00453

§ 371 Date: Apr. 17, 2007

(30) Foreign Application Priority Data
Aug. 4, 2004 (GB) 0417357.1

(51) Int. Cl. A61Q 13/00; A61K 8/06; A61K 8/58; A61Q 5/02; A61K 8/891

(52) U.S. Cl. 510/122; 510/158; 510/159

(57) ABSTRACT

A fluid personal care product, such as a shampoo or a liquid soap, comprising a fragrance, which fragrance is at least partially dissolved in a silicone oil. Preferably the silicone oil-soluble components of the fragrance are pre-dissolved in silicone oil and added separately from the non-soluble components. The fragrance in such a product remains substantially longer on skin and hair than is the case with conventional products with the same fragrance.

This invention relates to a method of depositing a fragrance on to a human substrate and to a fluid composition for use therein.

A fluid cleaning or care product (by which is meant a liquid, a gel or a cream) that imparts an enduring fresh odour to a human substrate (by which is meant skin or hair) is desirable, as this is perceived by customers as an indication of continuing freshness and cleanliness. This is more important in some areas than in others. For example, fragrance longevity in hair wash products needs to be longer than that of skin wash products, as most consumers do not use hair wash products daily. After a wash with a conditioning shampoo, it is desirable to have a perception of the fragrance typically for about 24 h-36 h (corresponding to the time between two washes). However, it is also desirable to have an enduring fresh odour with a skin wash or care product, such as a shower gel or a liquid soap.

Numerous attempts have been made to achieve this desirable goal. Some have involved the use of silicone oil emulsions. Silicone oil is widely used in personal care and fabric care products. In hair care products such as shampoo and conditioners, silicone oil and silicone gum (or mixtures thereof.) are very well known as keratin fibre cosmetic modifiers. The most common way to introduce silicone oil into a shampoo preparation is via a silicone-in-water emulsion. Such emulsions can be easily made, and some are available commercially as ready-to-use raw materials.

In the past, fragrance has simply been added to preformed emulsions and the mixture then added to products, such as shampoos. The problem is that it is difficult to introduce fragrances (nearly all of which are hydrophobic materials) into preformed emulsions. The fragrance may remain outside the emulsion, or it may destabilize it. The result may be that only the silicone oil deposits on the substrate, and not the perfume.

It has now been found that it is possible to provide a silicone emulsion that combines the good properties of silicone oil with a long-lasting deposition of fragrance. The invention therefore provides a fluid personal care product comprising a fragrance, which fragrance is at least partially dissolved in a silicone oil.

The invention further provides a method of depositing a fragrance on a human substrate, by applying to the substrate a fluid personal care product comprising an aqueous emulsion of silicone oil, the silicone oil having fragrance dissolved therein.

By “silicone oil” is meant any liquid silicone known to be useful in personal care products. One common (and preferred) silicone oil is polydimethylsiloxane. This material is commonly used in the making of shampoos, because it is known to be especially good at depositing on hair. However, any other suitable silicone oil may also be used, and the skilled person can readily provide such an oil. The preferred silicone oils for use in this invention are linear polydimethylsiloxanes having a molecular weight of between 5970 to 116500 g/mol. Mixtures of such polydimethylsiloxanes may also be used.

The linear polydimethylsiloxane is additionally characterized by a viscosity from 100cst to 60000cst, preferably from 2000 cst to 15000 cst. All viscosities mentioned herein are measured at 25° C. by means of a Brookfield RVT Viscometer. Spindles and speeds appropriate to the particular viscosity were used, as is the well-known practice in the art; for example, Spindle No. 5 and speed 20 were used for measurements in the preferred range between 6000 and 12000 centipoise.

A preferred silicone oil is a solution of polydimethylsiloxane gum in polydimethylsiloxane fluid. In an especially preferred version of this oil, the viscosity of the gum is greater than 1,000,000 centistokes, the viscosity of the fluid is from 10 to 100,000 centistokes and the ratio of gum to fluid is from 30:70 to 70:30, preferably from 40:60 to 60:40.

A simple method to determine the solubility of fragrance raw materials in silicone is to determine it experimentally. However, in view of the vast number of perfumery materials available and since they can be mixed in various proportions to form a complete fragrance, it is desirable to have a theoretical method of determining the solubility of perfumery raw materials into silicone.

One such method is the Hansen solubility parameter (HSP) method, which is useful in predicting the solubility of perfumery materials. HSP may be evaluated as disclosed, for example, in “Hansen Solubility Parameters: A User's Handbook” (Charles M. Hansen, CRC Press, 2000) or/and by using a software available on the market, such as Molecular Modeling Pro from www.Chemistry-Software.com or Hansen Solubility from Dynacomp Software.

It has been found that fragrance materials having a total HSP in the range of from 14-20, more preferably from 15-18 and most preferably from 15-16 are suitable for use in this invention. The predicted values correlate very well with experimentally-determined values. These values are subject to an error of +10%; this is a known and accepted feature of the Hansen method.

There are three Hansen empirically- and theoretically-derived parameters, a dispersion-force component (δd), a polar component (δp) and a hydrogen-bonding component (δh). Solubility Parameter units are given in MPa^{1/2}.

In a preferred embodiment, in addition to compliance with the total HSP as hereinabove described, the individual components should comply with the following values: δ_{d} from 14.5-18 (MPa)^{1/2}, δₚ from 0-9.5 (MPa)^{1/2} and δₕ from 2.92-11.5(MPa)^{1/2}

Examples of fragrance materials having suitable HSPs include (but are not restricted to) the following materials: propyl acetate, 2-ethylhexyl acetate, bornyl acetate, butyl acetate, dimethyl benzyl carbinol acetate, cis- & trans-hexenyl acetate, menthanyl acetate, neryl acetate, adoxal, allyl amyl glycolate, bergamote Givco 104, cedarwood essential oil china, boisambrene forte, irisone pure, isoraldeine 95, isoraldeine 40, isopulegol, methylionanthene, metambrate, ethyl amyl ketone, nutmeg essential oil, neroli ess, paracresyl methyl ether, ethyl oenanthate, isoamyl propionate, petitgrain ess Paraguay, isobutyl salicylate, rhubaflor, sauge offmalicis ess, terpinolene, undecavertol, toscanol, givescone, Iso E™ Super, geranyl acetate, hexyl acetate, dipentene, Galaxolide™.

The fragrance may be completely soluble in the silicone oil. However, in most cases, fragrances are blends of individual components and some of these components are silicone oil-soluble and others are not. In such a case, for the working of this invention, those components of the fragrance that are silicone oil-soluble are separated from the others and dissolved in the silicone oil. The remainder of the fragrance components is added to the personal care product formulation at some different time from that of the addition of the silicone oil solution.

The invention therefore further provides the use of a silicone oil in the deposition of fragrance on a human substrate silicone-soluble fragrance, in which silicone oil that part of the fragrance that is soluble in silicone has been dissolved.

There are several ways of incorporating the fragrance-containing silicone oil into a fluid product. One is simply to dissolve the fragrance material or materials in the silicone oil and then emulsify this oil into water, using a surfactant or a blend of surfactants. Emulsification may be carried out in any commercially-available high-speed shear mixer. Another way is to add the fragrance-containing silicone oil to a surfactant phase (comprising some or all of the surfactant necessary for the particular fluid product) and then blending that with the other ingredients to give the fluid product.

The surfactant or blend of surfactants may be anionic surfactants, nonionic surfactants or amphoteric surfactants or mixtures thereof.

Preferred types of anionic surfactants that are useful in the invention include:

carboxylates (soaps) such as ethoxy carboxylates, ester carboxylates; isothionates and taurates; phosphates (ethoxylates, alcohols, amides), sarcosinates (amides sarcosinates), sulfates: alcohol, alcohol ether, alkanolamides ethoxylates, natural oils, alkylphenol ethers; sulfonates: alcohol ether (ethane) or alkyl phenyl ether, paraffin, alkyl benzene, fatty acids and esters, naphthalene derivatives; olefin sulfonates, petroleum sulfonates, sulfosuccinates and sulfosuccinamate.

Other anionic surfactants useful for this emulsification include alkyl and alkyl ether sulfates, such as TEA-lauryl sulfate or sodium lauryl sulfate, ammonium lauryl sulfate, sodium laureth-2 sulfate or sodium laureth-3 sulfate, ammonium laureth-2 sulfate or ammonium laureth-3 sulfate, triethylamine lauryl sulfate, triethylamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, and sodium dodecyl benzene sulfonate, sodium N-lauroyl-L-glutamate, triethanol N-lauryoyl-L-glutamate, sodium N-lauroyl-N-methyl taurate, sodium N-lauroyl-N-methyl-.beta.-aminopropionate, and mixtures thereof.

The main types of nonionic surfactants that are useful in the invention are: alcohol ethoxylates, monoalkanolamides ethoxylates, fatty amine ethoxylate, fatty acid ethoxylate, ethylene oxide / propylene oxide copolymers, aryl phenol ethoxylates alkylpolyglucosides, the condensation products of ethylene and/or propylene oxide, sucroses esters and fatty amines oxide etc.

Especially preferred nonionic surfactants include blends of surfactants with HLB values between 6 and 16, preferably between 9 and 14.

The main types of amphoteric surfactants that are useful in the invention are alkylaminoacid salts, imidazoline products and betaines such as cocamidopropylbetaine and laurylbetaine.

In another preferred embodiment of the invention, the fragrance is introduced into a product in two components, one component, which is silicone-insoluble, being introduced directly into the product and the other (silicone-soluble) component into the silicone. That component introduced into the product will come out in the wet stage and that in the silicone in the dry stage.

The invention is further described by reference to the following non-limiting examples, which describe preferred embodiments.

### Preparation of Premix

A premix of silicone oil and soluble fragrance raw materials is first made. This is then added surfactant under slow stirring, and water is then added slowly while continuously mixing in order to form the emulsion.

15 g of each of three types of silicones, DC200 Fluid 100cst, DC200 Fluid 1000cts and DC200 Fluid 12500cst from Dow Coming corporation, are stirred for 10 minutes at a stirring rate of 1500 rpm to produce a homogeneous liquid. To each of these is added 20 g of the silicone oil-soluble components of a fragrance, and the mixture stirred again. This blend of silicone and fragrance is mixed with a blend of two surfactants, lauryl alcohol ethoxylated at 3 moles (70%) and a lauryl alcohol ethoxylated at 23 moles (30%) in a proportion of 5% of the surfactant blend. Water is then added slowly at a stirring rate of 2000 rpm. This emulsion is able to pass easily through a homogenizer at a pressure of 800 bars.

### EXAMPLE 1

Liquid shampoo formulations of the following compositions are prepared:

| | Sample 1 | Sample 2 | |
|---|---|---|---|
| Ammonium Laureth-3 sulfate (70%) | 14.00 | 14.00 | Primary Surfactant |
| Ammonium Lauryl Sulfate (70%) | 3.00 | 3.00 | Secondary Surfactant |
| Cocamidopropylbetaine (30%) | 4.00 | 4.00 | Amphoteric Surfactant |
| Cocamide MEA | 1.00 | 1.00 | Foam Booster |
| Cetyl alcohol | 0.30 | 0.30 | Deposition Aid |
| Polyquaternium-10 (Ucare JR-30m) | 0.20 | 0.20 | Cationic Polymer |
| ethylene glycol distearate | 4.00 | 4.00 | Pearling agent |
| (liquid form) | | | |
| DC 1491 (Dow Corning Corp.) | 2.50 | 2.50 | large particle size silicone used |
| | | | as keratin modifier |
| Kathon CG | 0.10 | 0.10 | Preservative |
| Disodium EDTA | 0.10 | 0.10 | |
| Fragrance insoluble in silicone | 0.395 | 0.395 | |
| Fragrance soluble in silicone | 0.355 | 0.000 | |
| Fragrance silicone emulsion premix | 0.000 | 2.130 | |
| (as prepared above - contains 16.66% | | | |
| of fragrance)7 | | | |

| | |
|---|---|
| Water | qsp 100 |
| Salt | qs to adjust viscosity |
| Citric acid | qs to adjust pH at 6.4-6.6 |

In each sample, the total fragrance is 0.75% (active) of the total composition, and the proportions of silicone-soluble and silicone-insoluble components are identical at 0.355% and 0.395% respectively. In Sample 1, the silicone-soluble and silicone-insoluble components of the fragrance are added separately, but without pre-solution in silicone oil. In Sample 2, the silicone-soluble components are made into a premix as hereinabove described, and the fragrance is added in two parts, as hereinunder described.

### Procedure For Making A Shampoo With And Without Premix

Polyquaternium-10 is added into water at 74° C. under slow stirring until well solvated. Cocamidopropylbetaine is then added, followed by ammonium laureth-3 sulfate at 600 rpm stirring to avoid an accumulation of complex between polyquaternium-10 and ammonium laureth-3 sulfate. Ammonium lauryl sulfate is then added, followed by disodium EDTA, cocamide MEA and cetyl alcohol, sequentially and in that order. The mixture is then cooled down to between 34° C. and 40° C., and liquid pearling agent is added. Slow stirring at 200 rpm is maintained until the mixture has cooled to room temperature, and then are added in sequence DC 1491 and the fragrance silicone emulsion premix followed by the silicone-insoluble fragrance part.

In case of the example without the fragrance silicone emulsion premix, both parts of the fragrance are mixed together and introduced as normal fragrance.

### Hair Swatch Wash Protocol

Fresh hair swatches(about 12 g each) are first washed 3 times with a solution of ammonium laureth-3 sulfate (70%) at 14% and cocamidopropylbetaine (30%) at 4% in water. They are then dried at ambient temperature for 48 hrs.

The hair swatches are each washed with about 1.5g of Illustration Shampoo samples 1 and 2.

### Protocol For Washing

1. Hair swatch is immersed into warm water for 10 seconds to wet thoroughly

2. 1.5g of shampoo is applied to each swatch by gentle massage between fingers for 2 minutes

3. The hair swatch is allowed to equilibrate with shampoo for 1 more minute

4. The swatch is rinsed under warm tap water for 45 sec to remove the foam.

5. It is sponged with a clean towel to remove the rest of the water

### Evaluation

### Sensory Evaluation

Each hair swatch is evaluated by a panel of 15 trained panelists.

Extraction from hair swatches: Perfume deposited on hair swatches was extracted by pentane in a recycled circuit by refluxing for 8 h. A standard (cyclohexyl chloride) is added to the pentane, the solvent is concentrated and injected into a GC-MS for identification and measurement of perfumery raw materials deposited.

### Comparison of Perfume Deposition Between Illustration Sample 1 A and 2

### Wet Stage

Sample 1: 203 μg/g of hair

Sample 2: 269 μg/g of hair

This shows that the deposition of fragrance raw materials by means of the composition according to the invention (using silicone-solubilised fragrance) is higher by 132% than that of the same fragrance introduced without dissolving in silicone oil.

### Dry Stage 24 H Natural Drying (Means Without Using Air Dryer)

Sample 1: 46 μg/g of hair

Sample 2: 174 μg/g of hair

In this case, the deposition of fragrance components by means of silicone-solubilised fragrance is higher by 378% than the same fragrance introduced without first dissolving in silicone oil.

### Overall Olfactive Evaluation After 24 H Natural Drying

A trained panel (15 persons) evaluated the intensity of the fragrance in the hair swatch.

Notation (0=no smell to 10=very strong fragrance smell)

### Comparison of Hair Swatch of Samples 1 And 2

Illustration Sample 1: Mean score=4.16

Illustration Sample 2: Mean score=6.24

### Dry Stage (3 Min Blow Drying)

Sample 1: 148 μg/g of hair

Sample 2: 178 μg/g of hair

Deposition of fragrance raw materials by means of silicone-solubilised fragrance is higher by 19.5% than deposition of the same fragrance introduced first dissolving in silicone oil. Less fragrance is retained with blow drying, because the higher temperatures and lower partial pressure involved cause more fragrance to be lost as a result of the vaporization of water and entrapment by its vapor.

These examples clearly show that fragrance raw material deposition is higher when delivered by means of fragrance dissolved in silicone oil in comparison with the same fragrance introduced without being dissolved in silicone.

For comparison, the whole fragrance (both silicone-soluble and -insoluble parts) was incorporated into a silicone emulsion in a conventional manner and the blow drying repeated. The results observed were inferior to either of the results given above.

### EXAMPLE 2

Shampoo formulations as shown below are prepared, as described in Example 1:

| | Sample 3 | Sample 4 | |
|---|---|---|---|
| Sodium Laureth-3 sulfate (70%) | 10.00 | 10.00 | Primary Surfactant |
| Sodium Lauryl Sulfate (70%) | 5.00 | 5.00 | Secondary Surfactant/Detergent |
| Cocamidopropylbetaine (30%) | 6.00 | 6.00 | Mild Amphoteric Surfactant |
| Cocamide MEA | 2.00 | 2.00 | Foam Booster/Viscosity Modifier |
| Cetyl alcohol | 0.30 | 0.30 | Deposition Aid |
| Ucare JR-30M | 0.20 | 0.20 | Cationic Gum |
| silicone emulsion* | 2.50 | 2.50 | |
| Fragrance insoluble in silicone | 0.395 | 0.395 | |
| Fragrance soluble in silicone | 0.355 | 0.000 | |
| Fragrance Silicone Emulsion | 0.000 | 2.130 | |
| premix (16.66% of fragrance) | | | |
| Preservatives | qs | | |
| Disodium EDTA | 0.10 | | |

| | |
|---|---|
| Water | qsp 100 |
| Salt | qs to adjust viscosity |
| Citric acid | qs to adjust pH at 6.4-6.6 |

*DC1491 ex Dow Corning

The proportions of overall fragrance and of silicone-soluble and -insoluble fragrance components are identical to those of Example 1.

The following wet stage results are found:

### Wet Stage

Sample 3: 390 μg/g of hair Sample 4: 3254 μg/g of hair

Analysis by GC-MS after extraction with pentane showed a deposition increase of 834% of silicone-solubilised fragrance. In the case of particular fragrance components, deposition of Linalool increased by a factor of 45, Iso E Super™ by 12, Galaxolide™ by 45 times and cedryl methyl ether by 11.

1. A fluid personal care product comprising a fragrance, which fragrance is dissolved in a silicone oil.

2. A product according to claim 1 in which the silicone oil is at least one polydimethylsiloxane, preferably a linear polydimethylsiloxane.

3. A product according to claim 2, in which the polydimethylsiloxane has a molecular weight of from 5970 to 116500 g/mol.

4. A product according to claim 2, in which the viscosity of the polydimethylsiloxane is from 100cst to 60000cst, preferably from 2000 centistokes to 15000 centistokes, as measured at 25° C. by means of a Brookfield RVT viscometer.

5. A product according to claim 2, in which the silicone oil is a polydimethylsiloxane gum dissolved in a polydimethylsiloxane fluid.

6. A product according to claim 5, in which the viscosity of the gum is greater than 1,000,000 centistokes, the viscosity of the fluid is from 10 to 100,000 centistokes and the ratio of gum to fluid is from 30:70 to 70:30.

7. A product according to claim 1 in which the fragrance that is soluble in the silicone oil has a total Hansen solubility parameter of from 14 to 20.

8. A product according to claim 7 in which the total Hansen solubility parameter has the following individual components, δ_{d} between 14.5-18 (MPa)^{1/2}, δ_{b} between 0 and 9.5 (MPa)^{1/2} and δₕ between 2.92 and 11.5(MPa)^{1/2}.

9. A method of making a fluid personal care product, comprising the step of incorporating into the product a fragrance dissolved in a silicone oil.

10. A method according to claim 9, in which the fragrance dissolved in the silicone oil is part of the total fragrance present in the product.

11. A method according to claim 9, in which the silicone oil in which the fragrance is dissolved is first emulsified into a blend of surfactants to be used in the product.

12. Use of a silicone oil in the deposition of fragrance on a human substrate silicone-soluble fragrance, in which silicone oil that part of the fragrance that is soluble in silicone has been dissolved.

13. A product according to claim 3, in which the viscosity of the polydimethylsiloxane is from 100cst to 60000cst, preferably from 2000 centistokes to 15000 centistokes, as measured at 25° C. by means of a Brookfield RVT viscometer.

14. A product according to claim 6, in which the viscosity of the gum is greater than 1,000,000 centistokes, the viscosity of the fluid is from 10 to 100,000 centistokes and the ratio of gum to fluid is from from 40:60 to 60:40.

15. A product according to claim 7 in which the fragrance that is soluble in the silicone oil has a total Hansen solubility parameter of from 15 to 18.

16. A product according to claim 15 in which the fragrance that is soluble in the silicone oil has a total Hansen solubility parameter of from 15 to 16.
